(19) United States
(12) Patent Application Publication
(10) Pub. No.: US 2020/0393377 A1
(43) Pub. Date: Dec. 17, 2020

(54) Microscopy for Rapid Antibiotic Susceptibility Test Using Membrane Fluorescence Staining and Spectral Intensity Ratio

(71) Applicant: POCARED Diagnostics LTD., Rehovot (IL)

(72) Inventor: Moshe Ben-David, Tel Aviv (IL)

(21) Appl. No.: 16/890,050

(22) Filed: Jun. 2, 2020

Related U.S. Application Data
(60) Provisional application No. 62/859,890, filed on Jun. 11, 2019.

(51) Int. Cl. G01N 21/64; C12Q 1/18; G01N 1/40; G01N 1/30

(52) U.S. Cl. CPC G01N 21/6428; C12Q 1/18; G01N 1/4077; G01N 2021/6421; G01N 2001/4088; G01N 2021/6439; G01N 1/30

(57) ABSTRACT

Single dye fluorescent staining, microscopic imaging, and the combination of differences in both intensity and spectral emission permit determination of the minimum concentration of an antibiotic needed to inactivate bacteria (Minimum Inhibitory Concentration (MIC)), thereby providing a means for rapid Antibiotic Susceptibility Testing (AST). By use of microscopic imaging, such as confocal imaging, this allows for a quick and easy means for clinicians to determine a suitable treatment regimen for patients suffering from bacterial infections, including those that eventually lead to sepsis.

### CROSS REFERENCE TO RELATED APPLICATIONS

This application claims the benefit of U.S. Provisional Patent Application No. 62/859,890, filed Jun. 11, 2019, which is incorporated herein by reference in its entirety.

### BACKGROUND OF THE INVENTION

### Field of the Invention

Rapid and reliable diagnostic and treatment methods are essential for effective patient care. Unfortunately, current antimicrobial susceptibility testing techniques generally require a prior isolation of the microorganism by culture (e.g., about 12 to about 48 hours), followed by a process that requires another about 6 to about 24 hours. For example, a confirmed diagnosis as to the type of infection traditionally requires microbiological analysis involving inoculation of blood cultures, incubation for 16-24 hours, plating the causative microorganism on solid media, another incubation period, and final identification 1-2 days later. Even with immediate and aggressive treatment, this can significantly affect a patient's prognosis, depending on the type of infection, and in some instances can lead to death.

Every hour lost before a correct treatment is administered can make a crucial difference in patient outcome. Consequently, it is important for physicians to determine what antibiotic(s) would be effective for the treatment. Given that current methods may take two days or more days to yield an answer, there is a strong need for a more rapid antibiotic sensitivity testing, preferably one that can identify specific antibiotic susceptibilities within only hours after blood samples are drawn. For instance, 1-3 hours, 1-5 hours, 1-10 hours, less than 24 hours, or after 24 hours, after blood samples are drawn. In another instance, 24 hours after blood samples are drawn and identified as being positive for a bacterial infection. A rapid test of this type would therefore permit physicians to initiate the optimal drug therapy from the start, rather than starting with a suboptimal or completely ineffective antibiotic, thereby greatly increasing clinical responsiveness.

Another issue encountered in treatment of patients with bacterial infections is a result of antibiotic resistance. Antimicrobial (i.e., antibacterial) resistance occurs when a microbe (i.e., bacteria and/or bacterial strain) acquires a genetic mutation, either spontaneously or by gene transfer, rendering it resistant to the treatment of one or more anti-bacterial agents, i.e., antibiotics. Drug-resistant organisms may acquire resistance to first-line antibiotics, necessitating the use of a second-line agent to which the microbe is sensitive. In the case of some bacterial strains that have gained resistance to multiple drugs, resistance to second- and even third-line antibiotics is sequentially acquired.

Resistance may take the form of a spontaneous or induced genetic mutation, or the acquisition of resistance genes from other bacterial species by horizontal gene transfer via conjugation, transduction, or transformation. Many antibiotic-resistance genes reside on transmissible plasmids facilitating their transfer. Antibiotic-resistance plasmids frequently contain genes conferring resistance to several different antibiotics.

The increasing rates of antibiotic-resistant bacterial infections seen in clinical practice stem from antibiotic use both within human and veterinary medicine. Any use of antibiotics can increase an evolutionary selective pressure in a population of bacteria, allowing resistant bacteria to thrive and non-resistant bacteria to die off. As resistance to antibiotics becomes more common, a greater need for alternative treatments arises. Antibiotic-resistance poses a grave and growing global problem to public health. With an increasing number of bacterial strains having resistance to antibiotics, individuals who require medicinal help are unable to acquire the proper treatment they require.

Therefore, in addition to determining the appropriate drug therapy, it is also crucial to determine the concentration/dosage of the drug therapy to be administered. Accordingly, it is an object of the present invention to provide: 1) quick, rapid determination of antibiotic susceptibility of a microbe, and 2) the minimum concentration needed for inhibition of the microbe(s).

Existing antimicrobial susceptibility testing (AST) techniques are lengthy processes. In general, current-day practice for identifying, isolating, and differentiating bacterial strains with and without antibiotic-resistance genes often involves a complex and lengthy process in microbiology labs. In the current processes, biological samples containing bacteria are first accepted into the lab. Systems like the BD Phoenix and bioMerieux Vitex 2 systems can be used to detect bacterial strains in manners known in the art. In another process, the biological samples are then streaked, using a sterilized loop, on agar plates containing a nutritionally-rich medium (for example, lysogeny broth or any other suitable broth). This agar plate contains spots that have been treated with an antibiotic. Once the specimen has been streaked on the plate, the agar plate is placed into a dedicated incubator for a minimum of 12 hours. The agar plates are then periodically checked for bacterial colony growth. As would be appreciated by one of ordinary skill in the art, if the biological sample contains bacteria, then bacterial colony growth is expected on the spots not containing the antibiotic. If the bacteria has not acquired an antibiotic-resistance gene, growth on the spots containing the antibiotic is not expected. However, if the bacterial strain has acquired an antibiotic-resistance gene, colony growth will occur on the spots that have been treated with the antibiotic. See for example, commonly owned U.S. Patent Application Publication No. 2008/0220465.

In another process, biological samples, upon collection, are sorted, labeled, and then inoculated into glass, round-bottom test tubes containing blood agar medium, or any other suitable nutritionally-rich growth medium (e.g., lysogeny broth) using a sterilized loop. The specimens are then inserted into an incubator for a 12 to 24 hour period. The samples are then observed and screened for positive (i.e., containing bacteria) and negative (i.e., not containing bacteria) cultures. Samples that appear to contain positive cultures are processed in order to isolate and suspend the bacteria in a biochemical fluid. This process involves suspension, dilution, vortexing, and turbidity measurements resulting in biochemical waste products. The cultures are then subjected to a species identification and antibiotics susceptibility tests, which exposes the bacterial suspensions to multiple reagents. After another 6 to 24-hour incubation period, the findings are interpreted and reported by lab technicians. This entire process generally takes at least 11 or more steps and at least 50 hours to obtain specimen results, and the process is labor-intensive.

Other processes to differentiate and identify between bacterial species and/or strains involves various types of nucleic acid sequencing methods. Briefly, DNA sequencing is the process of determining the precise order of nucleotides within a DNA molecule. It includes any method or technology that is used to determine the order of the four bases—adenine, guanine, cytosine, and thymine—in a strand of DNA. In these methods, once a biological sample is obtained, the bacteria contained in the biological sample needs to first be amplified. In other words, the biological sample is first collected and is then used to inoculate a suitable bacterial growth medium (e.g., blood growth medium or lysogeny broth). The inoculated sample is then grown at appropriate conditions for 12-24 hours. Upon growth, bacterial cells are pelleted from the culture medium, lysed, and processed to extract the bacterial DNA. Bacterial DNA is then cleaned, purified, and placed in a DNA sequencer. The growth of the bacteria and isolation of the bacterial DNA not only requires reagents, but also produces bio-waste material, and is additionally a time-consuming process. Additionally, nucleic acid sequencing methods require the use of primer sequences. A primer is a strand of short nucleic acid sequences (generally about 10 base pairs) that serves as a starting point for DNA synthesis. It is required for DNA replication because the enzymes that catalyze this process, DNA polymerases, can only add new nucleotides to an existing strand of DNA. By requiring primer sequences, this method additionally requires some minimal knowledge of the type of bacterial strain. Sequencing, as indicated, can additionally be time consuming and expensive.

Once the microbe is identified, the patient is then treated with an antibiotic. In some cases, the initial concentration/dosage may not be effective, due to a variety of reasons, such as antibiotic resistance. As a result, by the time a patient receives the appropriate antibiotic, at the correct dosage, prognosis may be significantly hindered.

Therefore, in view of the foregoing, a rapid antimicrobial susceptibility testing method is required in order to quickly provide effective treatment to a patient in need thereof.

### SUMMARY OF THE INVENTION

A method of determining a minimum inhibitory concentration (MIC) of one or more bacteria in a sample is provided. The method comprises: preparing a plurality of bacterial suspensions in a plurality of receptacles; adding different amounts of an antimicrobial agent to two or more of the plurality of bacterial suspensions, thereby creating a plurality of suspensions comprising a combination of bacteria and antimicrobial agent; incubating the plurality of suspensions comprising a combination of bacteria and antimicrobial agent at a suitable temperature for a suitable period of time to produce a plurality of incubated suspensions comprising a combination of bacteria and an antimicrobial agent; adding a single membrane-associated dye to the plurality of incubated suspensions; illuminating the incubated suspensions comprising the dye with a light at a one or more excitation wavelength for the dye; obtaining microscopic images of the incubated suspensions comprising the dye at two emission wavelengths of the dye (e.g., a first image at a first emission wavelength, and a second image at a second emission wavelength); determining, with at least one processor, an intensity of emitted light at the two emission wavelengths of the dye for individual bacterial cells in each image; determining, with at least one processor, a MIC for bacteria in the sample based upon the SIR (spectral intensity ratio) of emitted light at the two emission wavelengths of the dye for individual bacterial cells in each image. Any of the preceding may be computer-implemented and/or automated.

The following numbered clauses describe various embodiments or aspects of the present invention:

Clause 1. A method of determining a minimum inhibitory concentration (MIC) of one or more bacteria in a sample comprising:

preparing a plurality of bacterial suspensions in a plurality of receptacles;

adding different amounts of an antimicrobial agent to two or more of the plurality of bacterial suspensions, thereby creating a plurality of suspensions comprising a combination of bacteria and antimicrobial agent;

incubating the plurality of suspensions comprising a combination of bacteria and an antimicrobial agent at a suitable temperature for a suitable period of time to produce a plurality of incubated suspensions comprising a combination of bacteria and antimicrobial agent;

adding a single membrane-associated dye to the plurality of incubated suspensions;

illuminating the incubated suspensions comprising the dye with light at a one or more excitation wavelength for the dye;

obtaining microscopic images of the incubated suspensions comprising the dye at two emission wavelengths of the dye;

determining, with at least one processor, an intensity of emitted light at the two emission wavelengths of the dye for individual bacterial cells in each image;

determining, with the at least one processor, an MIC for bacteria in the sample based upon the relative intensity of emitted light at the two emission wavelengths of the dye for individual bacterial cells in each image.

Clause 2. The method of clause 1, wherein the MIC is determined by:

determining a spectral intensity ratio (SIR) for each incubated suspension based upon the intensity of emitted light at the two emission wavelengths of the dye for individual bacterial cells in each image; and

determining the MIC based upon the spectral intensity ratios or spectral dead live ratios (SDLs), as a function of the antimicrobial concentration.

Clause 3. The method of clause 1, wherein the MIC is determined using a step function.

Clause 4. The method of clause 1, wherein the MIC is determined using a step function that is in the form of:

$$y ( x ) = a \cdot erf ( \frac{b π ( x - c )}{2} )$$

wherein a is a scaling parameter, b determines the step function slope, and c is the MIC value.

Clause 5. The method of clause 1, wherein the MIC is determined using a step function that is in the form of:
y(x)=a·tan h[b(x−c)], wherein a is a scaling factor, b determines the step function slope and c is the MIC; or
where y(x)=a·a tan[b(x−c)], wherein a is a scaling factor, b determines the step function slope and c is the MIC.

Clause 6. The method of clause 2, further comprising, with the at least one processor:

determining a dead/live (D/L) ratio of bacterial cells for each suspension from the intensity of emitted light at the two emission wavelengths of the dye for individual bacterial cells in each image;

calculating a Spectral-Dead-Live ratio (SDL) values by taking the spectral intensity ratios and multiplying them with the D/L ratios; and

determining the MIC based upon the SDL as a function of the antimicrobial concentration.

Clause 7. The method of clause 6, wherein the MIC is determined by plotting SDL as a function of the antimicrobial concentration.

Clause 8. The method of clause 6, wherein the MIC is the first derivative or second derivative of the SDL.

Clause 9. The method of any one of clauses 1-8, wherein the image is obtained using a confocal microscope.

Clause 10. The method of any one of clauses 1-9, wherein the single membrane-associated dye is a styryl dye or a cyanine dye.

Clause 11. The method of any one of clauses 1-10, wherein the single membrane-associated dye is N-(3-Triethylammoniumpropyl)-4-(4-(Dibutylamino)Styryl)Pyridinium Dibromide or N-(3-Triethylammoniumpropyl)-4-(6-(4-(Diethylamino) Phenyl) Hexatrienyl) Pyridinium Dibromide.

Clause 12. The method of any one of clauses 1-11, wherein the excitation wavelength is a wavelength selected between the range of 360 nm and 570 nm.

Clause 13. The method of any one of clauses 1-12, wherein the emission wavelength is a wavelength ranging from 520 nm to 850 nm.

Clause 14. The method of any one of clauses 1-13, wherein the sample is a bodily fluid.

Clause 15. The method of clause 14, wherein the sample is blood, plasma, serum, or urine.

Clause 16. The method of any one of clauses 1-13, wherein the sample is a clinical isolate.

Clause 17. The method of any one of clauses 1-16, wherein the suitable incubation temperature is between 35° C. and 40° C.

Clause 18. The method of any one of clauses 1-17, wherein the suitable period of incubation time is between 30 minutes and 5 hours.

Clause 19. The method of any one of clauses 1-18, wherein the varying concentrations of an antimicrobial agent are prepared by serial dilutions.

Clause 20. The method of any one of clauses 1-19, wherein bacteria of the sample are concentrated and diluted to a fixed concentration of bacteria to prepare the plurality of bacterial suspensions in a plurality of receptacles.

Clause 21. The method of clause 20, wherein the bacteria of the sample are concentrated by centrifugation or by filtration.

Clause 22. The method of clause 20, wherein the bacteria are diluted in a liquid growth medium.

Clause 23. The method of any one of clauses 1-22, further comprising after adding different amounts of the antimicrobial agent to two or more of the plurality of bacterial suspensions, removing a portion of each of the plurality of incubated suspensions comprising a combination of bacteria and an antimicrobial agent and placing each removed portion in a new receptacle.

Clause 24. The method of any one of clauses 1-23, further comprising determining the gram-type of the one or more bacteria in the sample prior to illuminating the incubated suspensions comprising the dye with a light at a one or more excitation wavelengths for the dye.

### BRIEF DESCRIPTION OF THE DRAWINGS

FIG. 1 is a flow diagram showing the overall process flow for the methods and systems described herein.

FIG. 2 provides photomicrograph images of E. coli cells, with a green filter (530 nm, left) and a red filter (610 nm, right), respectively.

FIG. 3 provides plots showing the SIR as a function of the antibiotic concentration for the InCell measurement (top) and flow cytometer measurement (bottom).

FIG. 4 provides a scatter plot showing the fluorescence intensity at a red wavelength (610 nm) vs. fluorescence intensity at a green wavelength (530 nm) for sample images at four antimicrobial concentrations.

### DESCRIPTION OF THE INVENTION

The following description is merely exemplary in nature and is in no way intended to limit the invention, its application, or uses. While the description is designed to permit one of ordinary skill in the art to make and use the invention, and specific examples are provided to that end, they should in no way be considered limiting. It will be apparent to one of ordinary skill in the art that various modifications to the following will fall within the scope of the appended claims. The present invention should not be considered limited to the presently disclosed aspects, whether provided in the examples or elsewhere herein.

The use of numerical values in the various ranges specified in this application, unless expressly indicated otherwise, are stated as approximations as though the minimum and maximum values within the stated ranges are both preceded by the word “about.” In this manner, slight variations above and below the stated ranges can be used to achieve substantially the same results as values within the ranges. Also, unless indicated otherwise, the disclosure of ranges is intended as a continuous range including every value between the minimum and maximum values. As used herein, “a” and “an” refer to one or more.

As used herein, the terms “comprising,” “comprise” or “comprised,” and variations thereof, are open ended and do not exclude the presence of other elements not identified. In contrast, the term “consisting of” and variations thereof is intended to be closed, and excludes additional elements in anything but trace amounts.

As used herein, the term “patient” or “subject” refers to members of the animal kingdom including, but not limited to, human beings, and “mammal” refers to all mammals, including, but not limited to, human beings.

As used herein, the term “sample” refers to a material to be tested or analyzed. The sample contains bacteria and may be obtained from various sources. For instance, the sample to be analyzed may be a liquid, semi-liquid, or dry sample. The sample may be obtained from drinking water, a food or a beverage, a pharmaceutical product, a personal care product, or a body fluid. Samples may be obtained from a municipal water system, a well, potable water, waste water, a natural water source, recreational water, or a soil. In different embodiments, samples are obtained from medical devices. Examples of medical devices include, but are not limited to, implants, patches and heart valves. In other instances, samples may be obtained from bodily fluids. These may include, but are not limited to, blood or plasma, saliva, urine, throat sample, or gastrointestinal fluid (these may also be referred to as “biological samples”). “Samples” may also refer to clinical isolates. Clinical isolates may, in some instances, refer to bacteria that was isolated from bodily fluids and stored by suitable laboratory means. In general, clinical isolates refer to isolated bacteria. Therefore, in short, the term “samples” most broadly refers to the presence (or speculated presence) of bacteria. In some instances, the sample may be bacteria isolated from a source (such as a clinical isolate), whereas in other instances the sample may refer to a substance carrying bacteria/microbial agents (such as blood, urine, water, etc.).

As used herein, the terms “bacteria” (bacterial or bacterium) and “microbe” (microbial) refer to the same thing. That is, they refer to single-cell, prokaryotic, microorganisms, they are small, usually rod or cocci shaped, and may be disease causing. Bacteria-causing diseases are typically treated with antibiotics. Additionally, “bacterial strain” or “bacterial isolates,” refer to the same thing. Further, as recited herein “clinical isolate” refers to the same thing as a “bacterial isolate.” That is, a strain/isolate is a genetic variant, or subtype, of a bacterium. In other words, one type of bacterial species may contain several different strains. The strains differ based on genetic mutations, such as through acquisition of additional genes, such as antibiotic-resistance genes, etc. These terms would be understood by a person of ordinary skill in the art.

As used herein, the terms “antibacterial” and “antimicrobial” refer to the same thing. That is, they refer to anything that is capable of killing and/or inactivating a bacterial or microbial organism.

As used herein, “live cell,” “live bacteria,” or “active bacteria” means a bacterial cell which has the potential to grow and divide. “Dead” and “inactivated” are used interchangeably to refer to dead bacterial cells.

As used herein, the “treatment” or “treating” of a wound, defect, infection, or the like means administration to a patient by any suitable dosage regimen, procedure and/or administration route, an amount of a composition, device or structure effective to, and with the object of achieving a desirable clinical/medical endpoint, including attracting progenitor cells, healing a wound, correcting a defect, etc.

As used herein, “dosage regimen” means the schedule of doses of a therapeutic agent at a particular concentration, per unit of time, including the time between doses (e.g., every 6 hours) or the time when the dose(s) are to be given (e.g., at 8 a.m. and 4 p.m. daily), and the amount (that is, the concentration) of a medicine to be given at each specific time.

A “therapeutically effective amount” refers to an amount effective, at dosages and for periods of time necessary, to achieve the desired therapeutic result. An “amount effective” for treatment of a condition is an amount of an active agent or dosage form, such as the coacervate composition described herein, effective to achieve a determinable endpoint. The “amount effective” is preferably safe—at least to the extent the benefits of treatment outweighs the detriments and/or the detriments are acceptable to one of ordinary skill and/or to an appropriate regulatory agency, such as the U.S. Food and Drug Administration, CLSI or EuCAST. A therapeutically effective amount of a drug or dosage regimen may vary according to factors such as the disease state, age, sex, weight of the individual, and the ability of drug or dosage regimen to elicit a desired response in the individual. A therapeutically effective amount is also one in which any toxic or detrimental effects of drug or dosage regimen are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). For example, a single bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., and doses may be proportionally reduced or increased as indicated by the exigencies of the therapeutic situation. In some instances, it may be especially advantageous to formulate parenteral compositions in dosage unit form for ease of administration and uniformity of dosage. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

In some instances, as provided herein, a dosage regimen may mean the administration of one or more antibiotics and a specific concentration and at specific times to a patient in need thereof.

Fluorescence spectroscopy has been extensively exploited for studies of molecular structure and function in chemistry and biochemistry. However, its effectiveness in microbial identification and characterization has only been recognized in the last two decades.

Visible light is electromagnetic radiation falling generally in the wavelength range of from 380 nm to 780 nm, with “green” light falling generally in the wavelength range of from 475 nm to 570 nm, such as from 500 nm to 565 nm, or, for example, 530 nm, and “red” light falling generally in the wavelength range of from 590 to 780 nm, or, for example, 610 nm.

Briefly, as would be appreciated by one of ordinary skill in the art, fluorescence spectroscopy refers to a type of electromagnetic spectroscopy that analyzes fluorescence from a sample. It involves using a beam of light (for instance, ultraviolet light) that excites the electrons in fluorescent molecules of certain compounds and causes them to emit light; typically, but not necessarily, visible light. Light falling within wavelengths that excite the fluorescent molecules (e.g., a fluorescent dye) has an excitation frequency (λₑₓ) within the excitation spectrum for the fluorescent molecule. Light falling within wavelengths that the fluorescent molecule produce upon excitation has an emission frequency (λₑₘ) within the emission spectrum for the fluorescent dye.

“Imaging” refers to obtaining one or more images of a sample, such as a microscopic image in which individual cells are distinguishable either visually or by any suitable image analysis, computer program, process, software, application, algorithm, module, or the like. Images may be obtained either at one or more specific wavelengths, e.g., by using optical or digital filters, or over a broad range of wavelengths, including, e.g., both red and green lights. Digital images may be obtained by any useful method and device. Two types of sensors are broadly known and used for digital imaging; charge-coupled devices (CCDs) and complementary metal-oxide semiconductor (CMOS) sensors. Images may be processed as described herein using commercial or proprietary software. Images may be taken in succession at different wavelengths, e.g., using different optical or digital filtering.

Imaging may take place in microscope slides, optionally including wells for transfer of fixed volumes of incubated cells. Multi-well plates, as are broadly-known, may be used for high-throughput screening. For example, a 96-well microplate useful for high-content screening using fluorescent microscopy includes optically transparent well bottoms, such as polystyrene, cyclic olefin copolymer, or glass bottoms, such as Corning® HCS glass bottom microplates having flat, optically clear glass bottoms.

In one embodiment, an optical filter is used to obtain an image over a limited spectrum, such as within the emission spectrum of a fluorescent dye as described herein, such as ranging from 500 nm to 780 nm, or at a green wavelength (such as from 500 nm to 565 nm, or 530 nm) and at a red wavelength (such as from 590 to 780 nm, such as 610 nm). In a further embodiment, two images are taken in succession in which the first image is filtered digitally to produce a green image, and the second image is filtered digitally to obtain a red image, or vice-versa. In this manner, two images can be obtained in rapid succession or using two cameras each with a different filter, reducing bacteria shifts between images and the wavelength of the acquired images can be finely-tuned to obtain maximum resolution. A person of ordinary skill may choose suitable methods to use to determine spectral intensity of individual bacteria in an image at different wavelengths, including choice of optics and processing algorithms.

Image processing may include a process or algorithm to identify, outline, or otherwise distinguish, and optionally map, individual cells in the image, e.g., defined by increased intensity or brightness, or different color, as compared to background, and quantifying intensity of fluorescent emission in any useful manner. In a broad-spectrum image, individual cells can be mapped to ensure emission intensity at red and green wavelengths is obtained from a single cell. Where the red and green images are obtained at different times, the individual cells may move, and as such, movement and mapping/tracking of the individual cells may prove more difficult, but suitable methods and tracking algorithms may be employed to track individual cells.

In one embodiment, a laser confocal imaging device is used to obtain one or more images of the bacteria culture. The device produces light in the form of a laser emitting within the excitation spectrum of the dye that is used in the assay described herein. For example, for Synaptogreen or FM 1-43, a suitable excitation wavelength is 480 nm. In one embodiment, a green optical filter (e.g., an optical band-pass filter) is inserted between the sample and the imaging sensor, passing light in the range of, for example, from 500 nm to 565 nm, such as 530 nm. Once the “green” image is obtained, the green optical filter is removed and is replaced by a “red” optical filter, passing orange to red light in the range of, for example, from 590 to 780 nm, such as 610 nm. In another embodiment, the images can be taken at the same time using 2 cameras, each with a different filter. The imaging system is not limited to confocal imaging, and other microscopic imaging systems may be used, such as other optical or lens-free microscopic imaging systems.

Provided herein are methods that exploit fluorescence spectroscopy allowing for the detection of live bacteria as compared to dead bacteria, and further allowing for the determination of the minimum concentration needed for an antibiotic to inhibit bacterial activity.

Specifically, provided herein is a method for determining the effectiveness of a dosage regimen and determining the minimum inhibitory concentration (MIC) of a dosage regimen. In general, the method includes: obtaining a sample containing bacteria; preparing a set of vessels, such as test tubes or 96 well plates (first receptacles) containing the sample; and incubating the vessels, such as test tubes or plates, containing the sample with a range of varying concentrations (such as, by serial dilutions) of an antimicrobial agent at a suitable temperature (such as, between 30° C. and 50° C., between 35° C. and 40° C., or around 37° C.) for a given amount of time (for instance, between 30 minutes and 5 hours or between 2 hours and 4 hours). After incubation, all or a portion of the incubated samples may be transferred to imaging-compatible vessels, such as optical cups, cuvettes, or wells of a multi-well dish (second or new receptacles). A suitable fluorescence dye is added to the bacteria, and the bacteria are subjected to optical analysis, wherein the optical analysis includes a microscope or microscopic fluorescent imaging system, such as a laser-based confocal imaging platform, and wherein the optical analysis includes exciting the fluid sample by illuminating the sample with light (electromagnetic radiation) at one or more excitation wavelengths of the dye, imaging the sample in a microscope; determining the ratio of intensity of emissions from at least two wavelengths of electromagnetic radiation, and thereby determining the spectral intensity ratio (SIR) and the ratio of live bacteria to dead bacteria; and based upon the ratio determining the minimum inhibitory concentration.

In some instances, where the sample is, for example, a bodily fluid, the method may first include the following: 1) obtaining the bodily fluid sample, 2) centrifuging the sample (for example, 15 minutes at 24×g), and 3) diluting the supernatant with a suitable broth (for example, Cation-Adjusted Mueller Hinton Broth (CAMHB)).

In some instances, where the sample may be a clinical isolate, the method may first include the following steps: 1) obtaining the clinical isolate, 2) streaking the clinical isolate on agar plates containing a suitable growth medium (e.g., blood agar plates), 3) incubating the plates overnight, e.g., at 37° C., and 4) picking single colonies and suspending them in a suitable buffered solution (for example, phosphate buffered saline) and adjusting the number of bacteria to a 0.5 McFarland standard.

The methods provided herein, in some instances, rely on serial dilutions in order to determine the minimum concentration needed for a specific antimicrobial agent to inactivate a given microbe. Serial dilution is well-known in the art and generally refers to the stepwise dilution of a substance in solution. Usually the dilution factor at each step is constant, resulting in a geometric progression of the concentration in a logarithmic fashion. As provided herein, serial dilutions of the antimicrobial agent permits the testing of a range of concentrations of the antimicrobial agent in order to determine if the antimicrobial agent is effective in inactivating the microbe and, if so, the minimum concentration needed to inactivate the microbe.

As provided herein, the given concentration of an antimicrobial agent may vary from microbe to microbe (if known), antimicrobial agent to antimicrobial agent, the presence of resistance genes in the microbe, or any other relevant factors. The concentration of the antimicrobial agent at each step of the serial dilution will vary based upon these factors, and others, and is not meant to be a limiting feature. For instance, the concentration of the antimicrobial agent may range from 0 μg/ml to 5 mg/ml, and all subranges therebetween inclusive. One example range of concentration of the antimicrobial may for instance be: 0 (control sample), 0.5 μg/ml, 1 μg/ml, 2 μg/ml, 4 μg/ml, 8 μg/ml, 16 μg/ml, 32 μg/ml, 64 μg/ml, 128 μg/ml, 256 μg/ml, and all subranges therebetween inclusive. Also, diluting can be greater than 256 μg/ml or smaller than 0.5 μg/ml.

The methods described herein use a fluorescent dye to stain the microbe cells. For instance, upon staining bacteria with fluorescent membrane dyes, such as a styryl dye, the emission fluorescence of live bacteria versus inactive bacteria are weaker and shifted. Such phenomena might be the result of the interaction of the dyes in the lipophilic membrane environment in the live cells versus the inactive cells where the dyes are inserted to the more hydrophilic environment of the cytoplasm.

Dyes include, but are not limited to, fluorescent dyes which incorporate into the lipid bilayer. Examples of fluorescent dyes include styryl dyes and cyanine dyes. Representative styryl dyes include FM® 1-43, FM® 1-43FX, FM® 4-64 and FM® 4-64FX, FM® 2-10 dye. Representative cyanine dyes include Cy2, Cy3, Cy3B, Cy3.5, Cy5, Cy5.5 and Cy7. FM 1-43 is N-(3-Triethylammoniumpropyl)-4-(4-(Dibutylamino)Styryl)Pyridinium Dibromide, purchased from Life Technology (#T-35356), and also sold by Sigma as “Synaptogreen” (#S6814). FM 4-64 is N-(3-Triethylammoniumpropyl)-4-(6-(4-(Diethylamino) Phenyl) Hexatrienyl) Pyridinium Dibromide purchased from Life Technology (#T-13320) or Sigma as “Synaptored” (#S6689).

More than one dye may be used, but the present method may be performed with a single dye. The use of a single dye not only simplifies the method, but reduces variability caused by the presence of two dyes.

Provided herein, in some instances, are methods for differentiating and comparing live bacteria from inactive bacteria. The method is performed using spectral intensity ratio (SIR) analysis. SIR measures the intensity of emitted light after excitation at two wavelengths, and obtaining the ratio of the emitted light between the two wavelengths. Specifically, upon excitation of a specimen at a specific wavelength, measurable differences are evident in both the maximum emission peak and emission intensity between live and inactive bacteria. Accordingly, the ratio of emission intensities at two designated wavelengths or spectral intensity ratio can be used as a means of differentiating live bacteria from inactive bacteria. It is believed that use of SIR does not depend on the amount of dye used and the number of cells because SIR relies on a ratio of intensities.

More specifically, the spectral intensity ratio (SIR) maybe determined as follows:

$$SIR = \frac{{I}_{λ 2}}{{I}_{λ 1}} = \frac{I 2}{I 1}$$

where I_{λ1}=I1=an emission intensity at a first wavelength, and

where I_{λ2}=I2=an emission intensity at a second wavelength, for example

$$S I R = \frac{{I}_{λ = 610}}{{I}_{λ = 530}}$$

Where “I” (intensity of emission) is the mean value of the scatter plot at each wavelength.

In this case, I_{λ=610} is the intensity at the 610 nm wavelength, and I_{λ=530} is the intensity at the 530 nm wavelength. I₂=I_{λ=610} and I₁=I_{λ=530} is preferable for Gram Negative bacteria and synaptogreen staining. Low SIR values correspond to active bacterium population, while high values show a larger inactive bacterium population. The I_{λ} may be at other wavelengths specifically for Gram Positive bacteria and SynaptoRed staining, wherein I2=I_{λ=780} and I1=I_{λ=670}. Also, the Gram Positive dyed bacteria may be illuminated at the one of the two wavelengths, for example, 488 nm or 532 nm. An appropriate dye is used for Gram Positive bacteria.

The method of the present invention, as noted above, may include removing a portion of a sample after incubation, transferring the portion of the incubated sample having different concentrations of an antimicrobial agent to cuvettes, or other suitable vessels; adding a suitable fluorescence dye to the portions; and then subjecting samples to an optical analysis to obtain SIR by microscopic imaging and image analysis. However, in some instances, the dye may be placed directly in the diluted sample having different concentrations of antimicrobial agents after incubation with the one antimicrobial agent.

The method of the present invention allows accurate and rapid differentiation of live cells from inactive cells through relying on excitation/emission at a single bacteria level-based analysis rather than culture based validation, as well as requiring the use of only one dye to successfully differentiate. Thus, as noted above, the method in some instances may include the steps of: staining the sample with a single membrane-associated dye; illuminating the sample with an incident light at excitation; measuring, for each bacterial cell (i) the intensity I1 of emitted light at wavelength λ1; and (ii) the intensity I2 of emitted light at wavelength λ2; and calculating a ratio I2/I1. In one embodiment, this may be done on a single cell and in another embodiment, this same process may be conducted for more than one cell. In further such embodiments, bulk intensity may be measured to determine whether a sample contains live or inactive bacteria. As noted above, optical analysis may be done in the same vessel used for incubation, or in a separate vessel. In embodiments, although the excitation wavelength may be preferably at 488 nm for a specimen for Gram Negative bacteria and 488 nm or 532 nm for Gram Positive bacteria, other excitation wavelengths may be used.

The emission spectrum profile may be measured with a spectral analyzer or emission filters. The excitation wavelength may be between about 360 nm and about 600 nm and the wavelengths at which I1 and I2 are measured may be between about 520 nm and about 800 nm. In one embodiment, for the dye FM 1-43, the excitation wavelength is 488 nm and the emission wavelengths at which I1 and I2 are measured are 530 nm and 610 nm, respectively. For the dye FM 4-64, the excitation wavelength could be in between 488 nm to 570 nm and the emission wavelengths at which I1 and I2 are measured are 670 nm and 780 nm, respectively. Spectral intensity ratio (SIR) calculation is done by dividing the mean fluorescence value of the red (e.g., 610 nm) channel by the mean fluorescence value of the green (e.g., 530 nm) channel. Typical values of SIR for active bacteria are lower than values for inactive bacteria, for example, between 0.7 and 2 for active bacteria, and >2.5 for inactive bacteria.

In some embodiments, the sample may be analyzed for success or failure of a bacterial inactivation treatment, such as (but not limited to) antibiotic or antibacterial treatment (also referred to herein as antimicrobial agent), chlorine inactivation, heating, ethanol, and UV irradiation. In further embodiments, a threshold value can be determined by taking the I2/I1 of a pretreatment sample and then compared to the I2/I1 of the sample to determine efficacy of the bacterial inactivation treatment.

After staining, the fluorescent intensity of the samples is determined, as described above, with fluorescence filters (for example, λ1=530/30 nm and λ2=616/30 nm) to determine and analyze the SIR. Additionally, events, e.g., population events, of the viable and non-viable bacteria, and the ratio between them (that is, Dead/Live), can be determined.

The data generated from the images can be plotted in a single dimension, to produce a histogram, or in two-dimensional dot plots or even in three dimensions. The regions on these plots can be sequentially separated, based on fluorescence intensity. We can separate among dead and live bacteria based on the average fluorescent intensity of both populations. Subsequently, a Dead/Live ratio can be determined based upon this analysis. The “Dead” portion of the ratio refers to the average intensity of the dead bacterial population, and the “Live” portion of the ratio refers to the average intensity of the live bacterial population.

The Dead/Live ratio (hereinafter “D/L”) is then multiplied by the calculated SIR index, as outlined above, thereby enhancing the resulting signal. Multiplication of the SIR by the D/L ratio is described as follows:

SDL=SIR*(Dead/Live)

As stated above, SIR may be plotted as a function of the antimicrobial concentration and approximated to a step function in order to determine the MIC Likewise, the SDL (Spectral-Dead-Live ratio) may also be plotted as a function of the antimicrobial concentration and approximated to a step function in order to determine the MIC.

Incorporation of the D/L ratio increases the differences between the dead and live populations thereby making it easier to determine the step function. When the step function is used with SIR alone the differences are smaller. Incorporation of SDL results in a larger step function thereby making it easier to determine MIC. Further, because the step function becomes larger, the effect of antibiotic treatment is easier to see and/or determine. Additionally, also as a result of the step function becoming larger, incubation time of the bacterial samples could be decreased. Overall, incorporation of SDL rather than SIR results in improved sensitivity and easier determination of MIC. An added advantage of the present invention is that by using ratios, the concentration of dye used should not affect the results of the analysis.

Like SIR, SDL can be used to determine if a bacteria is resistant to treatment. The SDL and SIR have the same behavior. The advantage of SDL over SIR is that it emphasizes the differences between the live and dead populations, and therefore the step function is clearer. As for determining if the bacteria are resistant or susceptible, it is done in the same manner as for the SIR. If the SDL behaves like a linear line with a slope smaller than a certain limit and its intercept is greater than the SDL of the control sample, the bacteria is susceptible to the antimicrobial agent. If the SDL behaves like a linear line, its slope is smaller than a certain value, and its intercept is of the same order of the control sample value, the bacteria is resistant to the antimicrobial agent.

Of note, the type of function used depends on the bacteria susceptibility. If the bacteria are resistant to the antimicrobial agent, the SIR plotted as a function of the antimicrobial agent concentration is a linear line almost parallel to the x axis, and is not a step function. This is also true if the medical guidelines for administrating the antimicrobial agent are above the bacterial MIC. A step function results only when the MIC falls within the antimicrobial concentrations values tested.

As recited herein, “minimum inhibitory concentration” or “MIC” refers to the lowest concentration of an antimicrobial (for example, an antibiotic) drug that will inhibit the visible growth of a microorganism after overnight incubation. It is believed that the specimens will have to be incubated in cultured media before the process to ensure that the proper antibiotic—bacteria interaction will take place.

As provided herein, MIC may be calculated by plotting the SIR as a function of the antimicrobial concentration and approximating it to a step function, for example, in the form of:

$$y ( x ) = a \cdot erf ( \frac{b π ( x - c )}{2} )$$

Where a, b and c are parameters and erf is the error function. The MIC is the value of the parameter c.

The values of the error function are as follows: y(x) is the SIR or the SDL. Both SIR and SDL are dimensionless physical values since they are ratios of the same physical quantity, in the case of SIR intensity. The parameter “a” is a scaling value and is also dimensionless, “b” determines the step slope and has the dimension of 1/(antibiotic concentration), and “c” is the x value where the error function equals to zero and has the dimension of antimicrobial concentration [μg/ml]. Other suitable functions may be used, for instance the tan h function. SIRs for different concentrations are determined on a case-by-case basis.

Other step functions may be utilized, such as, without limitation:
tan h, where y(x)=a·tan h[b(x−c)], wherein a is a scaling factor, b determines the step function slope and c is the MIC, or
arctan (a tan), where y(x)=a·a tan[b(x−c)], wherein a is a scaling factor, b determines the step function slope and c is the MIC.

Other suitable manners for determining MIC may be used. For example, one option would be to calculate the first derivative for each point (that is, for each SIR or SDL point). The MIC is the antimicrobial agent concentration corresponding to the maximum of the first derivative. Another option would be to use the following formula: (Max(SIR or SDL)+Min(SIR or SDL))/2 and find the value of the antimicrobial agent concentration corresponding to that value.

As indicated above, the present invention relates to an improvement over the current methods of utilizing membrane fluorescence staining and SIR to determine bacterial viability, minimum inhibitory concentration, and bacterial sensitivity or resistance to antibiotic treatment.

Bacteria may include, but are not limited to, Gram Negative bacteria and Gram Positive bacteria, such as coliform bacteria, enterobacteria, Salmonella, Listeria, Shigella, Pseudomonas, Staphylococcus or Methanobacterium. For instance, Escherichia coli, Klebsiella pneumonia, Acinetobacter, Proteus mirabilis, Enterococcus cloacae, Aeromonass, Klebsiella oxytoca, Enterobacter cloacae, Proteus mirabilis, and Citrobacter freundii.

Antibiotics (or antimicrobial agents) may include, but are not limited to, ampicillin, gentamicin, quinolones (e.g., ciprofloxacin), amoxicillin, carbapenems (e.g., imipenem), tetracyclines, chloramphenicol, ticarcillin, bactrim, etc.

In some aspects, the sample is initially filtered to isolate the bacteria in a concentrated form and is then diluted to a fixed concentration of bacteria. In other aspects, the sample is initially concentrated via centrifugation and then diluted to a fixed concentration of bacteria. Dilution of the concentrated bacteria may occur in a suitable medium, such as liquid growth medium.

The present invention is more particularly described in the examples that follow, which are intended to be illustrative only.

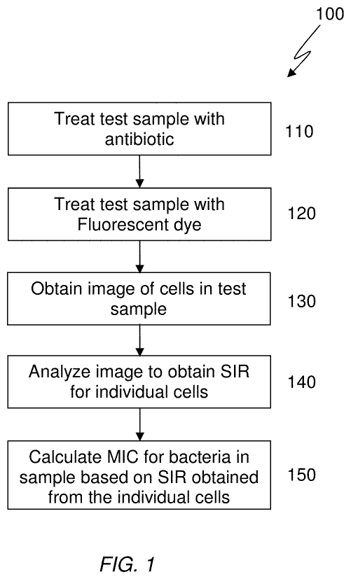

The overall process flow 100 for the methods provided herein is depicted in FIG. 1. Initially a bacteria sample is obtained. The sample may be from any source, such as a clinical sample from a patient, or an environmental sample. The bacteria is cultured by any suitable method in liquid medium, and may be diluted in liquid culture media, or other suitable diluents for culture purposes. Culture of the sample may be performed in any suitable vessel, such as a tube, flask, vial, or multi-well dish, as are generally-known in the microbiological arts. A dilution of the sample may be prepared by any suitable method, for example, per standard serial dilution methods, optionally according to any suitable laboratory standards or standard-operating-procedure. In one example, due to the nature of the assay described herein, at least three samples or aliquots are prepared, a first control with only culture medium, but no test sample, a second control with a reference bacteria sample, and the test sample. Multiple dilutions of the antimicrobial agent to be tested may be prepared by any useful method, e.g., by serial dilution of the antimicrobial agent. The process 100 may first comprise treating the test sample with an antibiotic 110 and incubating the test sample for a suitable time period, such as from 2-4 hours, to determine the effect of the antibiotic on any microbes, e.g., bacteria cells, in the test sample. Controls, including those without antibiotic, may be incubated with the test samples. The vessel in which the cells are incubated may be suitable for imaging, or optionally, each sample is transferred into an imaging vessel prior to imaging. An imaging vessel is a vessel suitable for holding the sample for imaging using a microscope imaging system as described herein, such as a chamber, slide, well, etc., for example, a 96-well plate, compatible with a microscope imaging system. Non-limiting examples of suitable imaging vessel, e.g., for high-content screening, include vessels with polystyrene, cyclic olefin copolymer or glass bottoms, such as Corning® HCS glass bottom microplates with flat, optically clear glass bottoms.

Next, either in the vessel in which the cells are cultured, or following transfer to the imaging vessel, the sample is treated with a suitable fluorescent dye 120, e.g., exposed to a fluorescent dye by addition of the dye to the sample. Examples of suitable fluorescent dyes are described herein. After treating the sample with a fluorescent dye, the sample, in the imaging vessel is exposed to light at a suitable excitation wavelength, e.g., as described herein, to produce fluorescence of cells in the sample. One or more images of the cells is obtained 130, and emission intensity of individual cells at two wavelengths, such as 610 nm and 530 nm, is obtained, and SIR is calculated 140 for each cell. SDL may be calculated. In examples, two images may be obtained with different optical filters, or output at two different wavelengths may be obtained using two cameras each with a suitable filter. The SIRs for each cell is then used to calculate 150 a suitable representation of the MIC for the cells, such as a plot of SIR versus antibiotic concentration. As described above, SIR is used to optionally calculate the SDL as part of determining the MIC. Alternatively, a scatter plot of samples may be prepared as output, with axes representing fluorescence intensity at two wavelengths, such as at 610 nm and at 530 nm.

Any of the data processing, comparing, and calculating activities described herein may be, and preferably are performed wholly or in part by use of a computing device, e.g., as computer-implemented methods, including, without limitation, calculating SIR, MIC, SDL, or D/L, image acquisition, image processing, statistical analysis, image comparison, graphing or plotting, producing output, etc. As used herein, the term “computing device” may refer to one or more electronic devices configured to process data. A computing device may, in some examples, include the necessary components to receive, process, and output data, such as a processor, a display, a memory, an input device, a network interface, and/or the like. Data is processed by the processor as directed by instructions, such as software and algorithms selected and configured to process the data in a desired manner, and as such the processor is programmed and/or configured to carry out a designated task. A computing device may be integrated into an imaging system, such as the GE IN Cell Analyzer systems (GE Healthcare Life Sciences), as described below. A computing device may be, for example, an optical device, a turnkey optical platform, a desktop computer, or other form of non-mobile computer, comprising suitable analytic software, such MATLAB, adapted to process data as described herein. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices.

A laser confocal imaging system, or a similar system, such as the GE IN Cell Analyzer system (GE Healthcare Life Sciences), e.g., IN Cell Analyzer 6000 with a variable aperture design, may be employed to obtain images of the samples, e.g., from a suitable 96-well microplate with well bottoms having sufficient optical quality for microscopy. Such devices often provide options for multiple excitation wavelengths, optical filters, and employ analytical software for high-throughput screening methods, such as those described herein. One non-limiting example of suitable analytical software is the IN Carta (GE Healthcare Life Sciences) image analysis software that can be implemented in connection with the GE IN Cell Analyzer systems. Other products, such as MATLAB, may be used to produce and analyze images as described herein.

As described above, separate images of the cells are obtained, at a first wavelength, such as 610 nm, and at a second wavelength, such as 530 nm. The images are then analyzed using a computer-implemented method, e.g., with software that can analyze intensity of individual cells in an image. A variety of computer systems, processes, algorithms, modules, etc. can be used to produce output representative of the proportion of cells that are alive and dead, and a suitable output, such as MIC, or other relevant AST output.

In one embodiment of a computer-implemented method for analyzing cells as described herein, an image of cells obtained by a method provided herein is received as input in any suitable file format. The image is analyzed to identify individual cells in the image, which will exhibit increased or decreased emission intensity at one or more wavelength, e.g., at a first wavelength, such as 610 nm, and/or at a second wavelength, such as 530 nm. One or more image parameters, such as white balance, hue, color balance, brightness, contrast, sharpness, etc. as are known in the imaging arts, may be adjusted as part of the analysis process to normalize the image, and a control sample, such as a test sample that is untreated with an antibiotic, or a sample that comprises a significant number of dead cells, may be used to normalize image values for further image processing. Normalization of the image may be conducted automatically in a computer-implemented process, but also can be done manually using suitable image processing software.

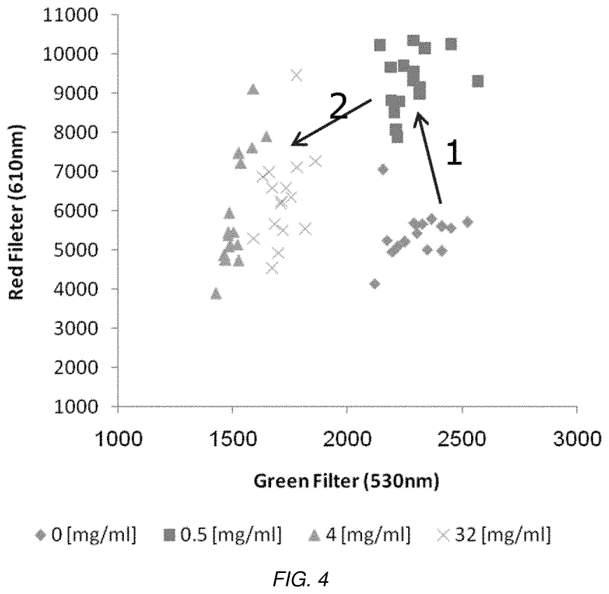

After normalization of the image(s), the emission intensity of individual cells, of a fixed number of cells, or of all cells in an image field is evaluated at a first wavelength, such as 610 nm, and at a second wavelength, such as 530 nm. The emission intensity of individual cells is measured independently at the first wavelength, such as 610 nm, and at the second wavelength, such as 530 nm. Intensity values at the first wavelength, such as 610 nm, and at the second wavelength, such as 530 nm, may then be used to calculate SIR, and, as described herein, SIR versus antibiotic concentration can be plotted or otherwise evaluated to determine an MIC of the antibiotic tested. Other computer-implemented methods, with suitable processes and/or algorithms can be used to evaluate MIC. For example, a clustering method can be used to classify cells in the test samples as being live or dead, and MIC can be determined based on a significant shift of a ratio of emission intensity at a red wavelength, such as 610 nm, and at a green wavelength, such as 530 nm (see, e.g., FIG. 4).

### EXAMPLES

### Example 1

In this example, a rapid method for AST and MIC determination directly from blood culture with a turnaround of less than 5 minutes, and less than 1 second per well, after 2-4 hours of antibiotics exposure is presented.

Materials and Methods:

In general, the method includes blood culture centrifugation, bacterial antimicrobial exposure for 2 to 4 hours, bacteria staining with a single fluorescence dye followed by laser confocal microscopy, and mathematical analysis.

Sample Preparation—Macro Dilution Method
a) Antimicrobial (Gentamicin) stock solutions were prepared according to CLSI recommendations, and were diluted in CAMHB, to a concentration which was 2 fold higher from the highest concentration recommended for each antimicrobial combination (“Class II Special Controls Guidance Document: Antimicrobial Susceptibility Test (AST) Systems”, Aug. 28, 2009, FDA).
b) For each sample/antimicrobial combination, a set of tubes was prepared for which lml of CAMHB medium was added, except for the first tube.
c) To each first tube of the set, 2 ml from the antimicrobial stock solution (b) was dispensed.
d) From the first tube a serial dilutions of 2 folds were prepared by transferring 1 ml of the solution to the next tube until the lowest required concentration by CLSI. 1 ml was discarded from the last tube in each set of tubes.
e) To each tube in the set, 1 ml of bacterial sample solution (a) was added.
f) For each serial dilutions set, two controls were added. As a negative control, a tube with 2 ml of CAMHB. As a positive control, a tube with bacterial sample solution (a) without antimicrobial agent.
g) All tubes were incubated at 37° C.
h) 2-4 hours from incubation initiation, 200 μl from each tube were transferred into a 96-well tissue culture plate, dyed with 2 μl of Synaptogreen or FM 1-43 (Sigma-Aldrich; Molecular Probes, respectively) and imaged using a GE Lifesciences IN Cell Analyzer 6000. Magnification was adjusted so that bacteria could be distinguished individually. The sample was excited using a 480 nm laser and 15 images were taken sequentially from each well. The images were taken through a green filter (530 nm) and red filter (615 nm) one after the other for each image.
i) images were analyzed using GE In Cell Investigator image analysis software. For each image the following operation were performed; segmentation for identifying the bacteria, for each bacteria the intensity in the green and yellow were recorded, and a scatter plot of the bacteria population was plotted (x axis—intensity in the green, y axis—intensity in the red).

### Data Interpretation:

To quantify the influence of a certain antimicrobial exposure we define the spectral intensity ratio (SIR) as follows:

$$spectral intensity ratio ( SIR ) = \frac{{I}_{λ = 610}}{{I}_{λ = 530}}$$

Where (I) is the mean value of the scatter plot at each wavelength. Low spectral intensity ratio (SIR) values correspond to active bacterium population, while high values show a larger inactive bacterium population. The main advantage of using a single dye and the above spectral intensity ratio (SIR) is the elimination of the result dependency on the dye concentration and optical efficiency.

The MIC is calculated by plotting the spectral intensity ratio (SIR) as a function of the antimicrobial concentration and approximating it to a step function in the form of

$$y ( x ) = a \cdot erf ( \frac{b π ( x - c )}{2} )$$

Where a, b, and c are parameters and erf is the error function. The parameters are used to approximate the measured SIR to a step function; a is a scaling parameter, b determines the step slope, and c is the MIC value, or a function of it. The SIR values are used to determine the erf parameters by a best fit approximation.

### Results:

We measured E. coli exposed to gentamicin at 4 concentration. The MIC was calculated using the spectral intensity ratio (SIR) calculation and step function estimation using MATLAB software.

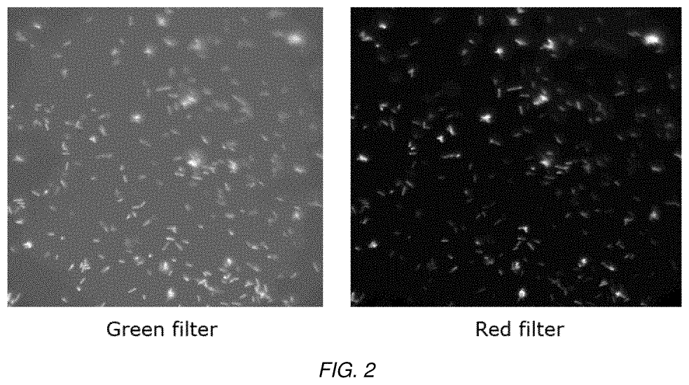

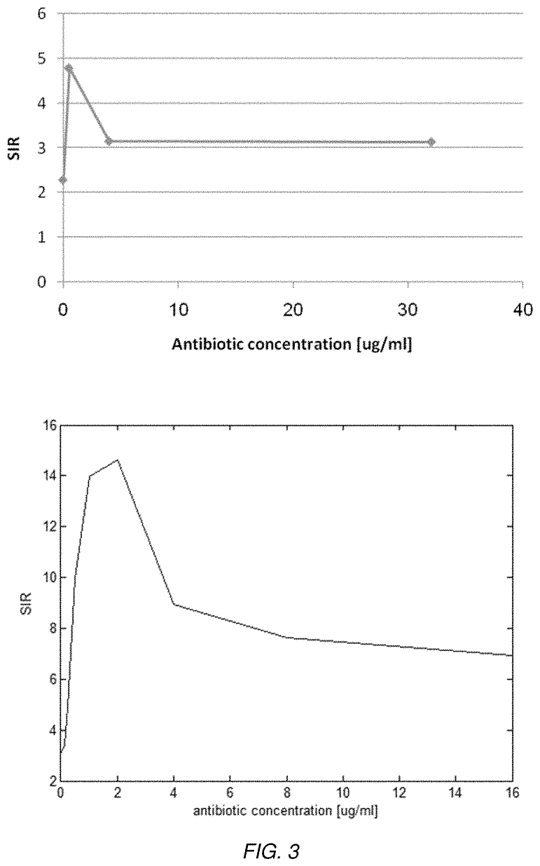

FIG. 2 shows examples of images of E. coli cells, with a green filter (left) and a red filter (right), respectively. FIG. 3 provides plots showing the SIR as a function of the antibiotic concentration for the InCell measurement (top) and flow cytometer measurement (bottom). As can been seen from the graphs, their shapes are similar, although the InCell measurement has less data points, and the MIC is 0.5 μg/ml in both cases. Live bacteria population (0 μg/ml) have lower SIR then the dead ones (>0.5 μg/ml). At the MIC (0.5 ug/ml) there is a large increase in the SIR. This is due to the appearance of a large inactive bacteria population. The SIR then decreases at very high (>4 ug/ml) antibiotic concentrations.

FIG. 4 provides a scatter plot showing the fluorescence intensity at a red wavelength (610 nm) vs. fluorescence intensity at a green wavelength (530 nm) taken from images of cells incubated with the indicated concentration of antibiotic. The live bacteria population (diamonds) is lower than the dead bacteria population (squares) near the MIC. The population shifts along arrow 1. At higher antibiotic concentrations (Triangles and X s) the dead population shifts to the lower intensities (arrow 2). This behavior was observed in the flow cytometer experiments as well.

In sum, using high content screening (GE InCell) it is possible to measure the spectral shift and determine whether the bacteria are live or dead. As such, the InCell system is a good candidate for rapid measurement of the SIR (e.g., less than 1 second per well).

While the present invention has been described in terms of the above examples and detailed description, those of ordinary skill will understand that alterations may be made within the spirit of the invention. Thus, the present invention is capable of many variations in detailed implementation, which may be derived from the description contained herein by a person of ordinary skill in the art.

The invention claimed is:

1. A method of determining a minimum inhibitory concentration (MIC) of one or more bacteria in a sample comprising:
preparing a plurality of bacterial suspensions in a plurality of receptacles;
adding different amounts of an antimicrobial agent to two or more of the plurality of bacterial suspensions, thereby creating a plurality of suspensions comprising a combination of bacteria and an antimicrobial agents;
incubating the plurality of suspensions comprising a combination of bacteria and an antimicrobial agent at a suitable temperature for a suitable period of time to produce a plurality of incubated suspensions comprising a combination of bacteria and an antimicrobial agent;
adding a single membrane-associated dye to the plurality of incubated suspensions;
illuminating the incubated suspensions comprising the dye with a light at a one or more excitation wavelength for the dye;
obtaining microscopic images of the incubated suspensions comprising the dye at two emission wavelengths of the dye;
determining, with at least one processor, an intensity of emitted light at the two emission wavelengths of the dye for individual bacterial cells in each image;
determining, with the at least one processor, an MIC for bacteria in the sample based upon the relative intensity of emitted light at the two emission wavelengths of the dye for individual bacterial cells in each image.

2. The method of claim 1, wherein the MIC is determined by:
determining a spectral intensity ratio for each incubated suspension based upon the intensity of emitted light at the two emission wavelengths of the for individual bacterial cells in each image; and
determining the MIC based upon the spectral intensity ratios or spectral dead live ratios (SDLs), as a function of the antimicrobial concentration.

3. The method of claim 1, wherein the MIC is determined using a step function.

4. The method of claim 1, wherein the MIC is determined using a step function that is in the form of:
$$y ( x ) = a \cdot erf ( \frac{b π ( x - c )}{2} )$$
wherein a is a scaling parameter, b determines the step slope, and c is the MIC value.

5. The method of claim 1, wherein the MIC is determined using a step function that is in the form of:
y(x)=a·tan h[b(x−c)], wherein a is a scaling factor, b determines the step function slope and c is the MIC; or
Y(x)=a·a tan[b(x−c)], wherein a is a scaling factor, b determines the step function slope and c is the MIC.

6. The method of claim 2, further comprising, with the at least one processor:
determining a dead/live (D/L) ratio of bacterial cells for each suspension from the intensity of emitted light at the two emission wavelengths of the dye for individual bacterial cells in each image;
calculating a Spectral-Dead-Live ratio (SDL) values by taking the spectral intensity ratios and multiplying them with the D/L ratios; and
determining the MIC based upon the SDL as a function of the antimicrobial concentration.

7. The method of claim 6, wherein the MIC is determined by plotting SDL as a function of the antimicrobial concentration.

8. The method of claim 6, wherein the MIC is the first derivative or second derivative of the SDL.

9. The method of claim 1, wherein the image is obtained using a confocal microscope.

10. The method of claim 1, wherein the single membrane-associated dye is a styryl dye or a cyanine dye.

11. The method of claim 1, wherein the single membrane-associated dye is N-(3-Triethylammoniumpropyl)-4-(4-(Dibutylamino)Styryl)Pyridinium Dibromide or N-(3-Triethylammoniumpropyl)-4-(6-(4-(Diethylamino) Phenyl) Hexatrienyl) Pyridinium Dibromide.

12. The method of claim 1, wherein the excitation wavelength is a wavelength selected between the range of 360 nm and 570 nm and/or the emission wavelength is a wavelength ranging from 520 nm to 850 nm.

13. The method of claim 1, wherein the sample is a bodily fluid.

14. The method of claim 13, wherein the sample is blood, plasma, serum, or urine.

15. The method of claim 1, wherein the sample is a clinical isolate.

16. The method of claim 1, wherein the suitable period of incubation time is between 30 minutes and 5 hours.

17. The method of claim 1, wherein the varying concentrations of an antimicrobial agent are prepared by serial dilutions.

18. The method of claim 1, wherein bacteria of the sample are concentrated and diluted to a fixed concentration of bacteria to prepare the plurality of bacterial suspensions in a plurality of receptacles.

19. The method of claim 18, wherein bacteria of the sample are concentrated by centrifugation or by filtration.

20. The method of claim 18, wherein the bacteria are diluted in a liquid growth medium.

21. The method of claim 1, further comprising after adding different amounts of the antimicrobial agent to two or more of the plurality of bacterial suspensions, removing a portion of each of the plurality of incubated suspensions comprising a combination of bacteria and antimicrobial agent and placing each removed portion in a new receptacle.

22. The method of claim 1, further comprising determining the gram-type of the one of more bacteria in the sample prior to illuminating the incubated suspensions comprising the dye with a light at a one or more excitation wavelength for the dye.
